(19) United States
(12) Patent Application Publication
(10) Pub. No.: US 2017/0196853 A1
(43) Pub. Date: Jul. 13, 2017

(54) USE OF AN INHIBITOR OF KINASE ACTIVITY, PARTICULARLY MASITINIB, FOR TREATMENT OF PROSTATE CANCER

(71) Applicant: AB SCIENCE, Paris (FR)

(72) Inventors: Alain MOUSSY, Paris (FR); Jean-Pierre KINET, Lexington, MA (US); Colin MANSFIELD, Ecully (FR)

(21) Appl. No.: 15/314,636

(22) PCT Filed: Jun. 2, 2014
(86) PCT No.: PCT/IB2014/001212

§ 371 Date: Nov. 29, 2016

(51) Int. Cl. A61K 31/496; A61K 31/7068; A61K 31/573; A61K 31/337

(52) U.S. Cl. CPC A61K 31/496; A61K 31/337; A61K 31/7068; A61K 31/573

(57) ABSTRACT

A treatment of patients afflicted with prostate cancer, wherein the treatment includes administering to a mammal in need thereof at least one tyrosine kinase inhibitor or mast cell inhibitor that is an inhibitor of kinase activity selected from the tyrosine kinases of: c-Kit, PDGFR, LYN, FYN, or any combination thereof, in particular masitinib, optionally in combination with at least one pharmaceutically active ingredient.

The present invention relates to a method for treating patients afflicted with prostate cancer, wherein said patients are treated with a tyrosine kinase inhibitor or mast cell inhibitor that is an inhibitor of kinase activity selected from the tyrosine kinases of: c-Kit, PDGFR, LYN, FYN, or any combination thereof, in particular masitinib, optionally in combination with at least one pharmaceutically active ingredient.

Global Incidence of Prostate Cancer

According to the United States National Cancer Institute's (NCI), prostate cancer is the second most common cancer and the second leading cause of cancer-related death in men in the United States. It is estimated that, in 2013, nearly 239,000 men will be diagnosed with prostate cancer in the United States, and nearly 30,000 men will die of the disease. Regarding the global burden, the International Agency for Research on Cancer (IARC) estimate that prostate cancer is the fourth most common cancer in both sexes combined and the second most common cancer in men. An estimated 1.1 million men worldwide were diagnosed with prostate cancer in 2012, accounting for 15% of the cancers diagnosed in men. With an estimated 307,000 deaths in 2012, prostate cancer is the fifth leading cause of death from cancer in men (6.6% of the total men deaths).

Current Therapies and Prognosis

Locally advanced prostate cancer is cancer that is starting to break out of the prostate, or has spread to the area just outside the prostate. Standard treatment for locally-advanced prostate cancer includes androgen-deprivation therapy (also referred to as hormone therapy), administered either on its own or together with surgical castration or radiotherapy (adjuvant therapy). Patients with locally advanced prostate cancer may be at high risk of recurrence, metastases and prostate-related death. In these patients, the addition of adjuvant androgen-deprivation therapy is now considered a standard of care for those men treated with radical radiotherapy [H Payne H, et al., British Journal of Cancer (2011) 105, 1628-1634. doi:10.1038/bjc.2011.385]. Adjuvant therapy is an additional cancer treatment given after the primary treatment to lower the risk that the cancer will come back. Adjuvant therapy may be given, for example, at an earlier disease stage than metastatic. An example of adjuvant therapy is an additional treatment given where detectable disease has been removed, but where there remains a statistical risk of relapse due to occult disease.

Androgen-deprivation therapy can be achieved by administering gonadotropin releasing hormone analogs (also known as Luteinizing-hormone-releasing hormone) or anti-androgens, which help control prostate cancer by stopping the hormone testosterone from reaching the prostate cancer cells. By itself, such androgen-deprivation agents do not cure cancer but can keep it under control and can also help to manage symptoms.

Advanced prostate cancer, also referred to as metastatic prostate cancer, is cancer that has spread from the prostate to other parts of the body. The standard medicinal treatment for advanced prostate cancer is androgen-deprivation therapy, achieved by administering gonadotropin releasing hormone analogs and anti-androgens. Such androgen-deprivation agents typically provide a median disease control of up to 22 months and overall survival of 28 to 36 months. Several types of androgen-deprivation agents are available including for example but not restricted to:
i) Gonadotropin releasing hormone agonists; for example, goserelin (Zoladex®), leuprorelin (Prostap®), triptorelin (Decapeptyl®).
ii) Gonadotropin releasing hormone antagonists; for example, degarelix (Firmagon®).
iii) Anti-androgens, for example, bicalutamide (Casodex®), flutamide (Chimax®, Drogenil®), cyproterone acetate (Cyprostat®), enzalutamide (Xtandi®).

Despite the majority of patients initially responding to androgen-deprivation therapy, for most patients the tumor eventually progresses and reaches a disease stage known as castration-resistant prostate cancer (CRPC), which is also referred to as castration-recurrent prostate cancer, hormonal-refractory prostate cancer, hormone-resistant prostate cancer, endocrine-resistant prostate cancer, or androgen-independent prostate cancer (AIPC). Castration-resistant prostate cancer includes a wide range of disease types: from patients without metastases or symptoms with rising prostate-specific antigen (PSA) levels despite androgen deprivation therapy to patients with metastases and significant debilitation due to cancer symptoms. Castrate-resistant prostate cancer is defined by disease progression despite androgen-deprivation therapy and may present as one or any combination of a continuous rise in serum levels of PSA, progression of pre-existing disease, or appearance of new metastases [Saad F, et al., Can Urol Assoc J. 2013 July-August; 7(7-8): 231-237]. Androgen receptor signaling remains essential for many prostate cancers that have progressed despite androgen deprivation therapy because even after medical or surgical castration, low levels of androgens are produced from nongonadal sources, such as the adrenal glands, at levels sufficient to activate androgen receptor mediated signaling.

Evidently therefore, there remains a need to develop new therapeutic agents or combination therapy regimens that improve survival of patients with locally advanced or advanced prostate cancer. Standard treatment for patients with CRPC includes additional treatment-lines with androgen-deprivation agents, chemotherapeutic agents (administered either as monotherapy or in combination with biological agents and/or corticosteroids), or immunotherapeutic and monoclonal antibody agents. Several types of treatment are available for CRPC patients including [Agarwal N, et al., Ann Oncol. 2014 Mar 20. (Epub ahead of print)]:
i) Androgen-axis inhibitors, more preferably androgen synthesis inhibitors and androgen receptor inhibitors; including for example but not restricted to abiraterone (Zytiga®), which is a novel selective cytochrome p450 17 inhibitor, and enzalutamide (Xtandi®), which is an androgen receptor antagonist with high binding affinity for the androgen receptor and inhibits nuclear translocation of the ligand-receptor complex.
ii) Chemotherapeutic agents including for example but not restricted to docetaxel (Taxotere®), cabazitaxel (Jevtana®), mitoxantrone (Novantrone®), estramustine (Emcyt®), doxorubicin (Adriamycin®), etoposide (VP-16); vinblastine (Velban®), paclitaxel (Taxol®), carboplatin (Paraplatin®), satraplatin, vinorelbine (Navelbine®), and gemcitabine.
iii) Steroids may be taken in combination with chemotherapeutic agents, for example but not restricted to dexamethasone, prednisolone, prednisone, and hydrocortisone.
iv) Immunotherapeutic and monoclonal antibody agents including for example but not restricted to sipuleucel-T (Provenge®), ipilimumab (Yervoy®), bevacizumab (Avastin®).

Docetaxel is the standard chemotherapy for men with CRPC. Often steroids such as prednisolone or dexamethasone, are administered in combination with chemotherapy to make chemotherapy more effective and lower the risk of side effects. Another treatment registered in CRPC is the immunotherapeutic agent sipuleucel-T for treatment of asymptomatic or minimally symptomatic metastatic CRPC. For patients with disease progression despite these treatments newer androgen-deprivation agents) such as androgen-axis inhibitors, more preferably androgen synthesis inhibitors and androgen receptor inhibitors (including abiraterone and enzalutamide) or chemotherapies such as cabazitaxel can be administered as the next treatment-line. Several other chemotherapeutic agents may be administered in subsequent treatment-lines or docetaxel combined with various other agents to improve the efficacy docetaxel. However, increased toxicity is often observed with such combination treatments that outweighs any benefits. A novel biological agent on which current clinical trials have focused is the combination of docetaxel-based therapy with small tyrosine kinase inhibitors (TKIs). Protein tyrosine kinases (PTKs) are rational therapeutic targets since they are often deregulated and closely linked to tumor growth and invasion and chemotherapy resistance. However, although several TKIs with different kinase selectivity have been investigated in combination with docetaxel or as single agents for treatment of CRPC, to date none have demonstrated sufficient therapeutic benefit for FDA approval and only a handful have progressed to phase 3 evaluation [Galsky 2010 Ann Oncol. 21 (11) 2135-2144]. For example, TKIs that have failed to show significant survival benefit against the trial's active comparator, or trials stopped early due to safety concerns include: imatinib [Galsky 2010 Ann Oncol. 21 (11) 2135-2144], sunitinib [Michaelson M D, et al., J Clin Oncol. 2014 Jan. 10; 32(2):76-82], lapatinib [Sridhar SS, et al. Am J Clin Oncol. 2010 December; 33(6):609-13], gefitinib [Pezaro C, et al., Am J Clin Oncol. 2009 August; 32(4):338-41], and dasatinib [Araujo J C, et al., Lancet Oncol. 2013 December; 14(13):1307-16].

Accordingly, there remains a high unmet medical need for improved therapeutic options with respect to efficacy and toxicity in locally advanced prostate cancer, advanced prostate cancer, unresectable prostate cancer, recurrent prostate cancer, metastatic prostate cancer and especially castrate-resistant prostate cancer or metastatic castrate-resistant prostate cancer.

### AIMS OF THE INVENTION

The invention aims to solve the technical problem of providing an active ingredient for the treatment of prostate cancer, including metastatic castrate-resistant prostate cancer, castrate-resistant prostate cancer, recurrent prostate cancer, unresectable prostate cancer, advanced prostate cancer, or locally advanced prostate cancer.

The invention also aims to solve the technical problem of providing an active ingredient for an efficient treatment of prostate cancer, including metastatic castrate-resistant prostate cancer, castrate-resistant prostate cancer, recurrent prostate cancer, unresectable prostate cancer, advanced prostate cancer, or locally advanced prostate cancer, especially in human male patients.

The invention also aims to solve the technical problem of providing an active ingredient that improves prior art methods for the treatment of prostate cancer, including metastatic castrate-resistant prostate cancer, castrate-resistant prostate cancer, recurrent prostate cancer, unresectable prostate cancer, advanced prostate cancer, or locally advanced prostate cancer.

The invention aims to provide an efficient treatment for prostate cancer, including metastatic castrate-resistant prostate cancer, castrate-resistant prostate cancer, recurrent prostate cancer, unresectable prostate cancer, advanced prostate cancer, or locally advanced prostate cancer at an appropriate dose, route of administration, and daily intake.

### SUMMARY OF THE INVENTION

Masitinib's main kinase target is c-Kit resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with an half inhibitory concentration (IC50) of 200±40 nM and blocking stem cell factor-induced proliferation and c-Kit tyrosine phosphorylation with an IC50 of 150±80 nM in Ba/F3 cells expressing human or mouse wild-type c-Kit. Stem cell factor (SCF), the ligand of the c-Kit receptor, is a critical growth factor for mast cells; thus, masitinib is an effective anti-mast cell agent, exerting a direct anti-proliferative and pro-apoptotic action on mast cells through its inhibition of c-KIT/SCF signaling [Reber L, et al. Eur J Pharmacol. 2006 Mar. 8; 533(1-3):327-40; and references therein]. In addition to its anti-proliferative properties against mast cells, masitinib can also regulate the activation of mast cells through its targeting of LYN and FYN, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FcεRI-mediated degranulation of human cord blood mast cells [Dubreuil et al., 2009, PLoS ONE; 4(9):e7258]. In vitro, enzymatic phosphorylation assays showed that masitinib inhibits LYN with an IC50 of 0.22 μM and FYN with an IC50 of 0.24 μM. Masitinib is also an inhibitor of PDGFR α and β receptors. Recombinant assays show that masitinib inhibits the in vitro protein kinase activity of PDGFR-α and β with IC50 values of 540±60 nM and 800±120 nM. In Ba/F3 cells expressing PDGFR-α, masitinib inhibited PDGF-BB-stimulated proliferation and PDGFR-α tyrosine phosphorylation with an IC50 of 300±5 nM [Dubreuil et al., 2009, PLoS ONE; 4(9):e7258].

Masitinib is differentiated from other TKIs not only in the tyrosine kinases it targets at clinically relevant doses but also by its high selectivity, which strongly implies it will exhibit a lower occurrence of off-target effects. Two large-scale independent studies have shown masitinib to have the highest selectivity from a wide range of protein kinase inhibitors, including all those approved or under clinical development at the time of publication [Anastassiadis T, et al., Nat Biotechnol. 2011 Oct. 30; 29(11):1039-45] [Davis M I, et al., Nat Biotechnol. 2011 Oct. 30; 29(11):1046-51].

In connection with the present invention, having discovered that masitinib indeed acts on prostate cancer, it would seem that a tyrosine kinase inhibitor or mast cell inhibitor that is an inhibitor of kinase activity selected from the tyrosine kinases of: c-Kit, PDGFR, LYN, FYN, or any combination thereof, in particular masitinib, optionally administered in combination with at least one pharmaceutically active ingredient, promotes survival in patients with prostate cancer via, but not limited to: modulation of mast cell activity through inhibition of the c-Kit signaling pathway with subsequent modulation of the tumor microenvironment and antimetastatic effects; and/or modulation of immunostimulation-mediated anticancer effects. Without wishing to be bound by the theory, the inventors consider that it is through at least one of these mechanisms of action that a compound of the invention, and in particular masitinib or a pharmaceutically acceptable salt thereof, can elicit a response in prostate cancer patients.

Data from a phase ½ clinical trial has provides proof-of-concept that there is a survival benefit associated with masitinib treatment of prostate cancer (see Example 1). This clinical trial was conducted in patients with a highly advanced and refractory stage of this disease (i.e. metastatic CRPC with failure to single-agent docetaxel) for whom there is the highest unmet medical need, as is common practice in fatal diseases. Furthermore, based on these findings there is also a likely survival benefit for patients with earlier stages of the disease (see Example 2) and especially those without metastatic disease.

Hence, in connection with the present invention, it would seem that despite the failure of several TKIs to show significant therapeutic benefit in the treatment of advanced prostate cancer, as single agents or in combination with other active therapeutic agents, surprisingly masitinib promotes survival in patients with prostate cancer, including patients in the most advanced and refractory stages of this disease, via one or more of the aforementioned mechanisms of action. Unexpectedly, without wishing to be bound by the theory, the inventors consider that it is through at least one of these mechanisms of action that a compound of the invention, and in particular masitinib or a pharmaceutically acceptable salt thereof, can elicit a response in prostate cancer patients.

The present invention relates to a method for the treatment of prostate cancer, wherein said method comprises administering to a mammal in need thereof at least one tyrosine kinase inhibitor or mast cell inhibitor that is an inhibitor of kinase activity selected from the tyrosine kinases of: c-Kit, PDGFR, LYN, FYN, or any combination thereof.

The present invention relates to a method for the treatment of prostate cancer selected from the group consisting of locally advanced prostate cancer, advanced prostate cancer, unresectable prostate cancer, recurrent prostate cancer, metastatic prostate cancer, and especially castrate-resistant prostate cancer or metastatic castrate-resistant prostate cancer, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib.

Definitions of the above disease states according to the National Cancer Institute (NCI) Dictionary of Cancer Terms are as follows (online resource: http://www.cancer.gov/dictionary; accessed 20 May 2014):
Locally advanced cancer is cancer that has spread from where it started to nearby tissue or lymph nodes.
Advanced cancer is cancer that has spread to other places in the body and usually cannot be cured or controlled with treatment.
Unresectable cancer is a cancer that is unable to be removed with surgery.
Recurrent cancer is cancer that has recurred (come back), usually after a period of time during which the cancer could not be detected. The cancer may come back to the same place as the original (primary) tumor or to another place in the body. Also called recurrence.
Metastatic cancer is cancer that has spread from the place where it first started to another place in the body. A tumor formed by metastatic cancer cells is called a metastatic tumor or a metastasis. The metastatic tumor contains cells that are like those in the original (primary) tumor.
Castrate-resistant prostate cancer is prostate cancer that keeps growing even when the amount of testosterone in the body is reduced to very low levels. Many early-stage prostate cancers need normal levels of testosterone to grow, but castrate-resistant prostate cancers do not.

The American Joint Committee on Cancer (AJCC) classifies prostate cancer according to a tumor, nodes, metastasis (TNM) classification system for prostate cancer [Prostate. In: Edge S B, Byrd D R, Compton C C, et al., eds.: AJCC Cancer Staging Manual. 7th ed. New York, N.Y.: Springer, 2010, pp 457-68].
TNM Stage III includes the criterion that ‘Tumor extends through the prostate capsule’. Hence, Stage III prostate cancer is broadly equivalent to locally advanced prostate cancer.
TNM Stage IV disease meets the primary tumor criterion that the ‘tumor is fixed or invades adjacent structures other than seminal vesicles such as external sphincter, rectum, bladder, levator muscles, and/or pelvic wall’. Hence, stage IV prostate cancer is broadly equivalent to advanced prostate cancer.
TNM Stage IV disease is also defined as any occurrence of metastasis in regional lymph node(s) or any occurrence of distant metastasis. Hence, stage IV prostate cancer is broadly equivalent to metastatic prostate cancer.
   (Definitions of TNM Stage III and IV are respectively taken from the NCI online resource: www.cancer.gov/cancertopics/pdq/treatment/prostate/HealthProfessional/Table3 and www.cancer.gov/cancertopics/pdq/treatment/prostate/HealthProfessional/Table4; accessed 20 May 2014).

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered to a human patient.

The expression “tyrosine kinase inhibitor or mast cell inhibitor that is an inhibitor of kinase activity selected from the tyrosine kinases of: c-Kit, PDGFR, LYN, FYN, or any combination thereof” covers notably also a single inhibitor inhibiting one, two, or three of PDGFR, LYN, FYN, and c-Kit including any combination thereof, and a single inhibitor inhibiting PDGFR, LYN, FYN, and c-Kit.

Methods for assessing a compound's ability to inhibit the function and activity of mast cells and assays for determination of said compound's effect on the activity of protein kinases has been described in the literature [Anastassiadis, T., S. W. Deacon, et al. Nat Biotechnol 29(11):1039-45] [Davis, M. I., J. P. Hunt, et al. Nat Biotechnol 29(11):1046-51] [Dubreuil et al., 2009, PLoS ONE 2009.4(9):e7258].

The expression “administering” covers notably the administration of a product independently of its physical or functional form, unless specified otherwise. This includes also the administration of a prodrug which metabolizes in vivo into at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib. This covers in particular the administration of one or more masitinib prodrug.

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered in combination with at least one pharmaceutically active ingredient. Said pharmaceutically active ingredient is preferably active in the treatment of locally advanced prostate cancer, advanced prostate cancer, unresectable prostate cancer, recurrent prostate cancer, metastatic prostate cancer, and especially castrate-resistant prostate cancer or metastatic castrate-resistant prostate cancer.

In one embodiment, said pharmaceutically active ingredient is a chemotherapeutic agent.

In one embodiment, said pharmaceutically active ingredient is a corticosteroid or a combination of corticosteroids.

In one embodiment, said pharmaceutically active ingredient is an androgen-deprivation agent.

In one embodiment, said pharmaceutically active ingredient is an immunotherapeutic agent.

In one embodiment, said pharmaceutically active ingredient is a monoclonal antibody agent.

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, when administered in association with one or more pharmaceutically active ingredients is administered to a human patient in the form of a pro-drug, which is metabolized in vivo into the pharmaceutically active drug.

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered in combination with at least one pharmaceutically active ingredient selected from the group consisting of androgen-deprivation agents, chemotherapeutic agents, corticosteroid agents, immunotherapeutic agents, monoclonal antibody agents, or any combination thereof.

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered in combination with at least one chemotherapeutic agent.

In one embodiment, said chemotherapeutic agent is selected from the group consisting of taxane agents, antimitotic agents, platinum-based antineoplastic agents, anthracycline antibiotic agents, anthracenedione antineoplastic agents, topoisomerase inhibitor antineoplastic agents, nucleoside analog agents, anti-tumor antibiotic agents, or antimicrotubule agents.

In one embodiment, said chemotherapeutic agent is selected from the group consisting of docetaxel, cabazitaxel, mitoxantrone, estramustine, doxorubicin, etoposide, vinblastine, paclitaxel, carboplatin, satraplatin, vinorelbine, and gemcitabine.

In one embodiment, said chemotherapeutic agent is docetaxel.

In one embodiment, said chemotherapeutic agent is cabazitaxel.

In one embodiment, said chemotherapeutic agent is gemcitabine.

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered in combination with at least one chemotherapeutic agent and at least one corticosteroid agent.

In one embodiment, said corticosteroid agent is selected from the group consisting of hydrocortisone type corticosteroids, for example hydrocortisone, hydrocortisone acetate, cortisone acetate, tixocortol pivalate, prednisolone, methylprednisolone, and prednisone, betamethasone type corticosteroids, for example betamethasone, betamethasone sodium phosphate, dexamethasone, dexamethasone sodium phosphate, and fluocortolone, and any combination thereof.

In one embodiment, said corticosteroid is prednisone.

In one embodiment, said corticosteroid is dexamethasone.

In one embodiment, said tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered in combination with at least one androgen-deprivation agent.

In one embodiment, said androgen-deprivation agent is selected from the group consisting of goserelin, leuprorelin, triptorelin, degarelix, bicalutamide, flutamide, cyproterone acetate, enzalutamide, and abiraterone.

In one embodiment, said androgen-deprivation agent is abiraterone.

In one embodiment, said androgen-deprivation agent is enzalutamide.

In one embodiment, tyrosine kinase inhibitor or mast cell inhibitor that is an inhibitor of kinase activity selected from the tyrosine kinases of: c-Kit, PDGFR, LYN, FYN, or any combination thereof, and in particular masitinib, is administered in combination with at least one immunotherapeutic or monoclonal antibody agent.

In one embodiment, said immunotherapeutic or monoclonal antibody agent is selected from the group consisting of sipuleucel-T, ipilimumab, or bevacizumab.

In one embodiment, said immunotherapeutic agent is sipuleucel-T.

In one embodiment, said monoclonal antibody agent is ipilimumab.

Preferably, said tyrosine kinase inhibitor is an inhibitor of kinase activity selected from the tyrosine kinases of: c-Kit, PDGFR, LYN, and/or FYN.

Preferably, said tyrosine kinase inhibitor or mast cell inhibitor that is an inhibitor of kinase activity selected from the tyrosine kinases of: c-Kit, PDGFR, LYN, FYN, or any combination thereof, is masitinib or a pharmaceutically acceptable salt thereof, and even more preferably, a masitinib mesilate salt.

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered at a daily dose of 1.0 to 12.0 mg/kg/day (mg per kg bodyweight per day).

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered at a dose of 2.5 to 9.5 mg/kg/day.

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered at a dose of 1.5, 3.0, 4.5, 6.0, 7.5, 9.0 or 10.0 mg/kg/day.

According to a preferred embodiment, masitinib is administered at a dose of 6 mg/kg/day in order to minimize toxicity.

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered once or twice a day. Preferably, said tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered twice a day.

According to a preferred embodiment, masitinib is administered at a dose of 6 mg/kg/day. For example: one dose of 3.0 mg/kg/day in the morning during breakfast, one dose of 3.0 mg/kg/day in the evening during dinner.

According to another preferred embodiment, masitinib is administered at a dose of 9 mg/kg/day. For example: one dose of 4.5 mg/kg/day in the morning during breakfast, one dose of 4.5 mg/kg/day in the evening during dinner.

In one embodiment, said tyrosine kinase inhibitor or mast cell inhibitor that is an inhibitor of kinase activity selected from the tyrosine kinases of: c-Kit, PDGFR, LYN, FYN, or any combination thereof, preferably masitinib, is dose escalated by increments of 1.5 mg/kg/day to reach a maximum of 9.0 mg/kg/day.

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is dose reduced by increments of 1.5 mg/kg/day to reach a minimum of 1.5 mg/kg/day.

Any dose indicated herein refers to the amount of active ingredient as such, not to its salt form.

Given that the masitinib dose in mg/kg/day used in the described dose regimens refers to the amount of active ingredient masitinib, compositional variations of a pharmaceutically acceptable salt of masitinib mesilate will not change the said dose regimens.

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered orally.

In one embodiment, tyrosine kinase inhibitor or mast cell inhibitor that is an inhibitor of kinase activity selected from the tyrosine kinases of: c-Kit, PDGFR, LYN, FYN, or any combination thereof, preferably masitinib, is administered in combination with said at least one pharmaceutically active ingredient in a combined preparation for simultaneous, separate, or sequential use.

Here, “simultaneous” means that the products are administered at the same time in the same formulation or composition. “Separate” means here that the products are administered, at the same time or sequentially, in separate formulation or composition.

In case of separate administration at the same time, administration is made in general within a few seconds or few minutes of each other, with generally no more than 30 minutes time difference between the administration of one of the pharmaceutical ingredients of interest and the administration of the other(s). Here, “sequential” means that there is a substantial time difference between the administration of one of the pharmaceutical ingredients of interest and the administration of the other(s), with generally more than 30 minutes time difference.

In one embodiment, the invention also relates to the combination of a tyrosine kinase inhibitor or mast cell inhibitor that is an inhibitor of kinase activity selected from the tyrosine kinases of: c-Kit, PDGFR, LYN, FYN, or any combination thereof, preferably masitinib, with at least one pharmaceutically active ingredient, preferably selected from the group consisting of (i) chemotherapeutic agents for example docetaxel, cabazitaxel, mitoxantrone, estramustine, doxorubicin, etoposide, vinblastine, paclitaxel, carboplatin, satraplatin, vinorelbine, gemcitabine; (ii) corticosteroid agents consisting of (iia) hydrocortisone type corticosteroids, for example hydrocortisone, hydrocortisone acetate, cortisone acetate, tixocortol pivalate, prednisolone, methylprednisolone, prednisone, (iib) betamethasone type corticosteroids, for example betamethasone, betamethasone sodium phosphate, dexamethasone, dexamethasone sodium phosphate, fluocortolone; (iii) androgen-deprivation agents for example goserelin, leuprorelin, triptorelin, degarelix, bicalutamide, flutamide, cyproterone acetate, enzalutamide, abiraterone; (iv) immunotherapeutic or monoclonal antibody agents for example sipuleucel-T, ipilimumab, bevacizumab; (v) or any combination thereof.

In one embodiment, a treatment of prostate cancer, especially castrate-resistant prostate cancer or metastatic castrate-resistant prostate cancer includes the administration of a combination of masitinib or a salt thereof (especially mesilate salt thereof) with docetaxel and prednisolone. In one embodiment, one or more other active ingredients according to the present invention may be administered according to such treatment.

In one embodiment, a treatment of prostate cancer, especially castrate-resistant prostate cancer or metastatic castrate-resistant prostate cancer, includes the administration of a combination of masitinib or a salt thereof (especially mesilate salt thereof) with gemcitabine. In one embodiment, one or more other active ingredients according to the present invention may be administered according to such treatment.

The invention also relates to a tyrosine kinase inhibitor or mast cell inhibitor that is an inhibitor of kinase activity selected from the tyrosine kinases of: c-Kit, PDGFR, LYN, FYN, or any combination thereof, preferably masitinib, as defined according to the present invention, for use in a treatment of prostate cancer, more particularly selected from the group consisting of locally advanced prostate cancer, advanced prostate cancer, unresectable prostate cancer, recurrent prostate cancer, metastatic prostate cancer, and especially castrate-resistant prostate cancer or metastatic castrate-resistant prostate cancer.

The invention also relates to a tyrosine kinase inhibitor or mast cell inhibitor that is an inhibitor of kinase activity selected from the tyrosine kinases of: c-Kit, PDGFR, LYN, FYN, or any combination thereof, preferably masitinib, as defined according to the present invention, for use in a treatment of prostate cancer, more particularly selected from the group consisting of locally advanced prostate cancer, advanced prostate cancer, unresectable prostate cancer, recurrent prostate cancer, metastatic prostate cancer, and especially castrate-resistant prostate cancer or metastatic castrate-resistant prostate cancer, in combination with at least one pharmaceutically active ingredient, preferably selected from the group consisting of (i) chemotherapeutic agents for example docetaxel, cabazitaxel, mitoxantrone, estramustine, doxorubicin, etoposide, vinblastine, paclitaxel, carboplatin, satraplatin, vinorelbine, gemcitabine; (ii) corticosteroid agents consisting of (iia) hydrocortisone type corticosteroids, for example hydrocortisone, hydrocortisone acetate, cortisone acetate, tixocortol pivalate, prednisolone, methylprednisolone, prednisone, (iib) betamethasone type corticosteroids, for example betamethasone, betamethasone sodium phosphate, dexamethasone, dexamethasone sodium phosphate, fluocortolone; (iii) androgen-deprivation agents for example goserelin, leuprorelin, triptorelin, degarelix, bicalutamide, flutamide, cyproterone acetate, enzalutamide, abiraterone; (iv) immunotherapeutic or monoclonal antibody agents for example sipuleucel-T, ipilimumab, bevacizumab; (v) or any combination thereof.

The invention also relates to a pharmaceutical composition or kit comprising a tyrosine kinase inhibitor or mast cell inhibitor that is an inhibitor of kinase activity selected from the tyrosine kinases of: c-Kit, PDGFR, LYN, FYN, or any combination thereof, preferably masitinib, for use in a method, according to the present invention, for the treatment of prostate cancer, more particularly selected from the group consisting of locally advanced prostate cancer, advanced prostate cancer, unresectable prostate cancer, recurrent prostate cancer, metastatic prostate cancer, and especially castrate-resistant prostate cancer or metastatic castrate-resistant prostate cancer, in combination with one or more pharmaceutically active ingredient, preferably selected from the group consisting of (i) chemotherapeutic agents for example docetaxel, cabazitaxel, mitoxantrone, estramustine, doxorubicin, etoposide, vinblastine, paclitaxel, carboplatin, satraplatin, vinorelbine, gemcitabine; (ii) corticosteroid agents consisting of (iia) hydrocortisone type corticosteroids, for example hydrocortisone, hydrocortisone acetate, cortisone acetate, tixocortol pivalate, prednisolone, methylprednisolone, prednisone, (iib) betamethasone type corticosteroids, for example betamethasone, betamethasone sodium phosphate, dexamethasone, dexamethasone sodium phosphate, fluocortolone; (iii) androgen-deprivation agents for example goserelin, leuprorelin, triptorelin, degarelix, bicalutamide, flutamide, cyproterone acetate, enzalutamide, abiraterone; (iv) immunotherapeutic or monoclonal antibody agents for example sipuleucel-T, ipilimumab, bevacizumab; (v) or any combination thereof.

Docetaxel is administered for example as follows: a daily dose of from 50 to 100 mg/m², preferably at 75 mg/m².

Prednisole, is administered for example as follows at a daily dose of from 5 to 20 mg, preferably 10 mg.

In one embodiment, docetaxel is administered for example at 75 mg/m² by intravenous infusion for example during 1 hour, once every 3 weeks (1 cycle=21 days) for 6 cycles, if possible for 8 to 10 cycles according to patient's tolerance, and prednisone at 5 mg is administrated continuously orally twice daily, wherein one cycle lasts 21 days.

Gemcitabine is administered for example as follows: intravenous infusion, preferably at 1000 mg/m² preferably within 30 minutes.

Abiraterone acetate is administered for example as follows: 1000 mg/day plus prednisone 5 mg twice daily.

Cabazitaxel is administered for example as follows: 25 mg/m² plus prednisone (5 mg/day).

Enzalutamide is administered for example as follows: 160 mg daily.

Mitoxantrone is administered for example as follows: 12 mg/m² once every 3 weeks

plus prednisone 10 mg/day.

Estramustine is administered for example as follows: 280 mg twice daily.

Sipuleucel-T is administered for example as follows: 3 complete doses, given at approximately 2-week intervals.

Ipilimumab is administered for example as follows: 10 mg/kg every 3 weeks.

Bevacizumab is administered for example as follows: 15 mg/kg every 3 weeks.

Leuprorelin is administered for example as follows: 3.75 mg once monthly; or 11.25 mg every 3 months.

Goserelin is administered for example as follows: 3.6 mg once monthly; or 10.8 mg every 3 months.

Bicalutamide is administered for example as follows: 50 mg orally daily for 10 days, started 3 days before LHRH agonist (leuprorelin or goserelin).

Flutamide is administered for example as follows: 250 mg three times daily for 10 days, started 3 days before LHRH agonist (leuprorelin or goserelin).

Degarelix is administered for example as follows: 240 mg as a loading dose, followed by 80 mg every 28 days starting 28 days after loading dose.

In one embodiment the invention relates to a method for the treatment of chemo-naïve metastatic castrate-resistant prostate cancer or chemo-naïve castrate-resistant prostate cancer, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof.

In one embodiment the invention relates to a method for the treatment of metastatic castrate-resistant prostate cancer or castrate-resistant prostate cancer after failure of at least one chemotherapeutic agent, wherein said method comprises administering to a mammal in need thereof, at least tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof. This includes second-line treatment of metastatic castrate-resistant prostate cancer or castrate-resistant prostate cancer.

In one embodiment the invention relates to a method for the treatment of metastatic castrate-resistant prostate cancer or castrate-resistant prostate cancer after failure of at two or more chemotherapeutic agents, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof. This includes third-line treatment of metastatic castrate-resistant prostate cancer or castrate-resistant prostate cancer.

The expression ‘failure of a chemotherapeutic agent’ covers notably a cancer that does not respond to treatment with said chemotherapeutic agent. The cancer may be resistant at the beginning of treatment or it may become resistant during treatment. Typically when this occurs, the treatment will need to be changed.

In one embodiment the invention relates to a method for the treatment of metastatic castrate-resistant prostate cancer or castrate-resistant prostate cancer after failed docetaxel-based chemotherapy, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof.

In one embodiment the invention relates to a method for the treatment of metastatic castrate-resistant prostate cancer or castrate-resistant prostate cancer in patients naïve to androgen-axis inhibitors, more preferably androgen synthesis inhibitors and androgen receptor inhibitors (including abiraterone and enzalutamide), wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof.

In one embodiment the invention relates to a method for the treatment of metastatic castrate-resistant prostate cancer or castrate-resistant prostate cancer after failure of at least one androgen-axis inhibitor, more preferably an androgen synthesis inhibitor or androgen receptor inhibitor (such as abiraterone or enzalutamide) , wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof. This includes second-line treatment of metastatic castrate-resistant prostate cancer or castrate-resistant prostate cancer.

In one embodiment the invention relates to a method for the treatment of metastatic castrate-resistant prostate cancer or castrate-resistant prostate cancer after failure of at two or more androgen-axis inhibitors, more preferably androgen synthesis inhibitors or androgen receptor inhibitors (such as abiraterone or enzalutamide), wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof. This includes third-line treatment of metastatic castrate-resistant prostate cancer or castrate-resistant prostate cancer.

The expression ‘failure of an androgen-axis inhibitor’ covers notably a cancer that does not respond to treatment with said androgen-axis inhibitor agent. The cancer may be resistant at the beginning of treatment or it may become resistant during treatment. Typically when this occurs, the treatment will need to be changed.

In one embodiment the invention relates to a method for the treatment of metastatic castrate-resistant prostate cancer or castrate-resistant prostate cancer in patients naïve to immunotherapeutic or monoclonal antibody agents, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof.

In one embodiment the invention relates to a method for the treatment of metastatic castrate-resistant prostate cancer or castrate-resistant prostate cancer after failure of at least one immunotherapeutic or monoclonal antibody agent, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof. This includes second-line treatment of metastatic castrate-resistant prostate cancer or castrate-resistant prostate cancer.

In one embodiment the invention relates to a method for the treatment of metastatic castrate-resistant prostate cancer or castrate-resistant prostate cancer after failure of at two or more immunotherapeutic or monoclonal antibody agents, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, optionally in combination with at least one pharmaceutically active ingredient, preferably selected from the group consisting of docetaxel, cabazitaxel, mitoxantrone, estramustine, doxorubicin, etoposide, vinblastine, paclitaxel, carboplatin, satraplatin, vinorelbine, gemcitabine, prednisone, dexamethasone, enzalutamide, abiraterone, sipuleucel-T, ipilimumab, bevacizumab, and any combination thereof. This includes third-line treatment of metastatic castrate-resistant prostate cancer or castrate-resistant prostate cancer.

The expression ‘failure of immunotherapeutic or monoclonal antibody agent’ covers notably a cancer that does not respond to treatment with said immunotherapeutic or monoclonal antibody agent. The cancer may be resistant at the beginning of treatment or it may become resistant during treatment. Typically when this occurs, the treatment will need to be changed.

In one embodiment the invention relates to a method for the treatment of advanced prostate cancer, metastatic prostate cancer, unresectable prostate cancer, or recurrent prostate cancer in patients naïve to androgen-deprivation agents, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, in combination with at least one pharmaceutically active ingredient, preferably selected from the group consisting of goserelin, leuprorelin, triptorelin, degarelix, bicalutamide, flutamide, cyproterone acetate, enzalutamide, abiraterone, and any combination thereof.

In one embodiment the invention relates to a method for the treatment of advanced prostate cancer, metastatic prostate cancer, unresectable prostate cancer, or recurrent prostate cancer after failure of at least one androgen-deprivation agent, wherein said method comprises administering to a mammal in need thereof, at least one tyrosine kinase inhibitor or mast cell inhibitor that is an inhibitor of kinase activity selected from the tyrosine kinases of: c-Kit, PDGFR, LYN, FYN, or any combination thereof, and in particular masitinib, in combination with at least one pharmaceutically active ingredient, preferably selected from the group consisting of goserelin, leuprorelin, triptorelin, degarelix, bicalutamide, flutamide, cyproterone acetate, enzalutamide, abiraterone, and any combination thereof. This includes second-line treatment of advanced prostate cancer, metastatic prostate cancer, unresectable prostate cancer, or recurrent prostate cancer.

In one embodiment the invention relates to a method for the treatment of advanced prostate cancer, metastatic prostate cancer, unresectable prostate cancer, or recurrent prostate cancer after failure of two or more androgen-deprivation agents, wherein said method comprises administering to a mammal in need thereof, tyrosine kinase inhibitor or mast cell inhibitor that is an inhibitor of kinase activity selected from the tyrosine kinases of: c-Kit, PDGFR, LYN, FYN, or any combination thereof, and in particular masitinib, in combination with at least one pharmaceutically active ingredient, preferably selected from the group consisting of goserelin, leuprorelin, triptorelin, degarelix, bicalutamide, flutamide, cyproterone acetate, enzalutamide, abiraterone, and any combination thereof. This includes third-line treatment of advanced prostate cancer, metastatic prostate cancer, unresectable prostate cancer, or recurrent prostate cancer.

The expression ‘failure of androgen-deprivation agent’ covers notably a cancer that does not respond to treatment with said androgen-deprivation agent. The cancer may be resistant at the beginning of treatment or it may become resistant during treatment. Typically when this occurs, the treatment will need to be changed.

In one embodiment the invention relates to a method for the treatment of locally advanced prostate cancer in patients naïve to androgen-deprivation agents, wherein said method comprises administering to a mammal in need thereof, tyrosine kinase inhibitor or mast cell inhibitor that is an inhibitor of kinase activity selected from the tyrosine kinases of: c-Kit, PDGFR, LYN, FYN, or any combination thereof, and in particular masitinib, as an adjuvant to radiotherapy in combination with at least one pharmaceutically active ingredient, preferably selected from the group consisting of goserelin, leuprorelin, triptorelin, degarelix, bicalutamide, flutamide, cyproterone acetate, enzalutamide, abiraterone, and any combination thereof. This includes adjuvant treatment of locally advanced prostate cancer.

The invention also relates to a pharmaceutical composition or kit comprising a tyrosine kinase inhibitor or mast cell inhibitor that is an inhibitor of kinase activity selected from the tyrosine kinases of: c-Kit, PDGFR, LYN, FYN, or any combination thereof, preferably masitinib, and at least one pharmaceutically active ingredient.

The invention also relates to the use of a tyrosine kinase inhibitor or mast cell inhibitor that is an inhibitor of kinase activity selected from the tyrosine kinases of: c-Kit, PDGFR, LYN, FYN, or any combination thereof, preferably masitinib, for the preparation of a medicament, or a pharmaceutical composition, for the treatment of prostate cancer, more particularly including metastatic castrate-resistant prostate cancer, castrate-resistant prostate cancer, recurrent prostate cancer, unresectable prostate cancer, advanced prostate cancer, or locally advanced prostate cancer, optionally in combination with at least one pharmaceutically active ingredient, preferably selected from the group consisting of (i) chemotherapeutic agents for example docetaxel, cabazitaxel, mitoxantrone, estramustine, doxorubicin, etoposide, vinblastine, paclitaxel, carboplatin, satraplatin, vinorelbine, gemcitabine; (ii) corticosteroid agents consisting of (iia) hydrocortisone type corticosteroids, for example hydrocortisone, hydrocortisone acetate, cortisone acetate, tixocortol pivalate, prednisolone, methylprednisolone, prednisone, (iib) betamethasone type corticosteroids, for example betamethasone, betamethasone sodium phosphate, dexamethasone, dexamethasone sodium phosphate, fluocortolone; (iii) androgen-deprivation agents for example goserelin, leuprorelin, triptorelin, degarelix, bicalutamide, flutamide, cyproterone acetate, enzalutamide, abiraterone; (iv) immunotherapeutic or monoclonal antibody agents for example sipuleucel-T, ipilimumab, bevacizumab; (v) or any combination thereof.

The terms “as defined according to the invention” or “according to the invention” refer to any embodiments or aspects of the invention alone or in combination without limitation, including any preferred embodiments and variants, including any embodiments and features relating to tyrosine kinase inhibitor or mast cell inhibitor that is an inhibitor of kinase activity selected from the tyrosine kinases of: c-Kit, PDGFR, LYN, FYN, or any combination thereof, preferably masitinib; the method of treatment of prostate cancer, including metastatic castrate-resistant prostate cancer, castrate-resistant prostate cancer, recurrent prostate cancer, unresectable prostate cancer, advanced prostate cancer, or locally advanced prostate cancer; pharmaceutical composition or kit comprising at least one pharmaceutically active ingredient, preferably selected from the group consisting of (i) chemotherapeutic agents for example docetaxel, cabazitaxel, mitoxantrone, estramustine, doxorubicin, etoposide, vinblastine, paclitaxel, carboplatin, satraplatin, vinorelbine, gemcitabine; (ii) corticosteroid agents consisting of (iia) hydrocortisone type corticosteroids, for example hydrocortisone, hydrocortisone acetate, cortisone acetate, tixocortol pivalate, prednisolone, methylprednisolone, prednisone, (iib) betamethasone type corticosteroids, for example betamethasone, betamethasone sodium phosphate, dexamethasone, dexamethasone sodium phosphate, fluocortolone; (iii) androgen-deprivation agents for example goserelin, leuprorelin, triptorelin, degarelix, bicalutamide, flutamide, cyproterone acetate, enzalutamide, abiraterone; (iv) immunotherapeutic or monoclonal antibody agents for example sipuleucel-T, ipilimumab, bevacizumab; (v) or any combination thereof.

“Masitinib” designates also an acceptable salt thereof for the intended use, especially masitinib mesilate, even if not explicitly stated.

“Pharmaceutically acceptable” by reference to a chemical compound has to be understood as including compounds that are physiologically acceptable, and more particularly those for which undesirable effects are acceptable in view of the pharmaceutical benefit.

The tyrosine kinase inhibitor or mast cell inhibitor that is an inhibitor of kinase activity selected from the tyrosine kinases of: c-Kit, PDGFR, LYN, FYN, or any combination thereof, preferably masitinib, and the optional at least one pharmaceutically active ingredient, are administered in a dosage regimen that comprises a therapeutically effective amount.

### DESCRIPTION OF THE INVENTION

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

In one embodiment, the tyrosine kinase inhibitor of the invention has the following formula [A]:

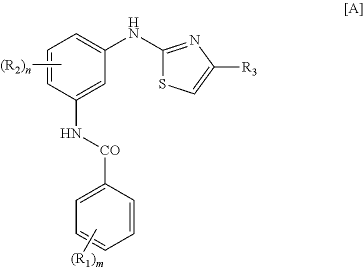

wherein R1 and R2, are selected independently from hydrogen, halogen, a linear or branched alkyl, cycloalkyl group containing from 1 to 10 carbon atoms, trifluoromethyl, alkoxy, cyano, dialkylamino, and a solubilizing group,

m is 0-5 and n is 0-4;

the group R3 is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;

(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;

or a pharmaceutically acceptable salt or solvate thereof.

Tyrosine kinase inhibitors of formula [A] can preferably be used as c-Kit inhibitors.

Unless otherwise specified, the below terms used herein are defined as follows:

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C6)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO2CH3; CONH2; OCH2CONH2; NH2; SO2NH2; OCHF2; CF3; OCF3; and such moieties may also be optionally substituted by a fused-ring structure or bridge, for example —OCH2O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR11R12, —NR13C(O)R14, a halo, —OR13, cyano, nitro, a haloalkoxy, —C(O)R13, —NR11R12, —SR13, —C(O)OR13, —OC(O)R13, —NR13C(O)NR11R12, —OC(O)NR11R12, —NR13C(O)OR14, —S(O)rR13, —NR13S(O)rR14, —OS(O)rR14, S(O)rNR11R12, —O, —S, and —N—R13, wherein r is 1 or 2; R11 and R12, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R11 and R12 taken together with the nitrogen to which they are attached is optionally substituted heterocycloalkyl or optionally substituted heteroaryl; and R13 and R14 for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown below, wherein the wavy line and the arrow line correspond to the point of attachment to core structure of Formula [A]:

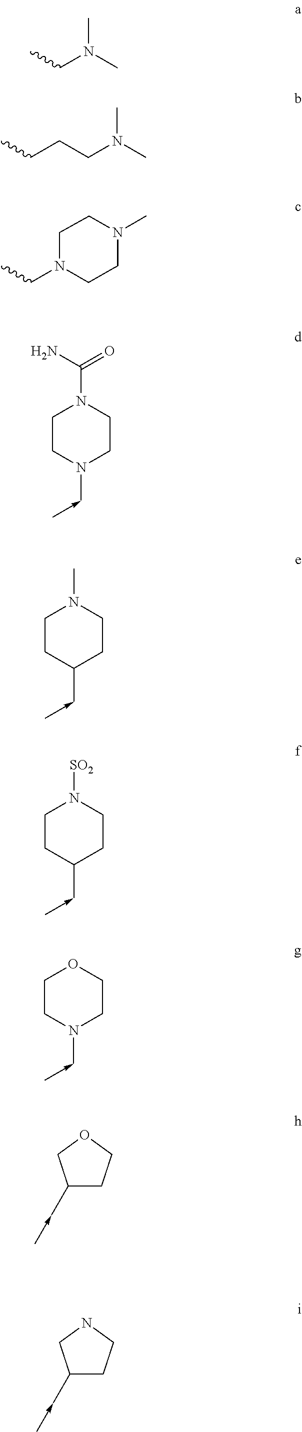

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

In a particular embodiment the tyrosine kinase inhibitor of the invention has general formula [B],

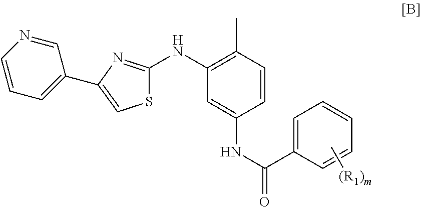

wherein:

R1 is selected independently from hydrogen, halogen, a linear or branched alkyl, cycloalkyl group containing from 1 to 10 carbon atoms, trifluoromethyl, alkoxy, amino, alkylamino, dialkylamino, solubilizing group, and m is 0-5.

In one embodiment, the tyrosine kinase inhibitor or mast cell inhibitor that is an inhibitor of kinase activity selected from the tyrosine kinases of: c-Kit, PDGFR, LYN, FYN, or any combination thereof, is masitinib or a pharmaceutically acceptable salt thereof, more preferably masitinib mesilate.

Masitinib is a c-Kit/PDGFR inhibitor with a potent anti-mast cell action.

New potent and selective c-Kit, platelet derived growth factor receptor (PDGFR) inhibitors are 2-(3-aminoaryl)amino-4-aryl-thiazoles described in AB Science's PCT application WO 2004/014903.

Masitinib is a small molecule drug, selectively inhibiting specific tyrosine kinases such as c-Kit, PDGFR, LYN, and FYN without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009.4(9):e7258] [Davis et al., Nat Biotechnol 2011, 29(11): 1046-51]. The chemical name for masitinib is 4-(4-methylpiperazin-1-ylmethyl)-N-[4-methyl-3-(4-pyridin-3y1thiazol-2-ylamino) phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO2008/098949.

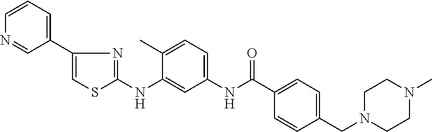 [SMILES: Cc1ccc(NC(=O)c2ccc(CN3CCN(C)CC3)cc2)cc1Nc1nc(-c2cccnc2)cs1]

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with an half inhibitory concentration (IC₅₀) of 200±40 nM and blocking stem cell factor-induced proliferation and c-Kit tyrosine phosphorylation with an 10₅₀ of 150±80 nM in Ba/F3 cells expressing human or mouse wild-type c-Kit. In addition to its anti-proliferative properties, masitinib can also regulate the activation of mast cells through its targeting of LYN and FYN, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FcεRI-mediated degranulation of human cord blood mast cells [Dubreuil et al., 2009, PLoS ONE; 4(9):e7258]. Masitinib is also an inhibitor of receptors PDGFR α and β. Recombinant assays show that masitinib inhibits the in vitro protein kinase activity of PDGFR-α and β with IC₅₀ values of 540±60 nM and 800±120 nM. In Ba/F3 cells expressing PDGFR-a, masitinib inhibited PDGF-BB-stimulated proliferation and PDGFR-α tyrosine phosphorylation with an IC₅₀ of 300±5 nM.

The present invention relates to a method for the treatment of CRPC in a mammal, and especially a human patient, wherein said method comprises administering to a human patient in need thereof, a tyrosine kinase inhibitor or mast cell inhibitor that is an inhibitor of kinase activity selected from the tyrosine kinases of: c-Kit, PDGFR, LYN, FYN, or any combination thereof, especially masitinib or a pharmaceutically acceptable salt thereof, optionally combined with at least one pharmaceutically active ingredient.

In relation to the present invention, the term “treatment” (and its various grammatical forms) refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

Advantageously, the use or method comprises a long term administration of an effective amount of said tyrosine kinase inhibitor or mast cell inhibitor that is an inhibitor of kinase activity selected from the tyrosine kinases of: c-Kit, PDGFR, LYN, FYN, or any combination thereof, especially masitinib or a pharmaceutically acceptable salt thereof, over more than 3 months, preferably more than 6 months.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

According to a particular embodiment, the composition of the invention is an oral composition.

In one embodiment, compositions according to the invention may be in the form of tablets.

In one embodiment, composition according to the invention may comprise from 50 to 500 mg of said tyrosine kinase inhibitor, mast cell inhibitor, PDGFR inhibitor, LYN inhibitor, or c-Kit inhibitor, especially masitinib or a pharmaceutically acceptable salt thereof. More particularly, the composition may comprise from 100 to 500 mg of said tyrosine kinase inhibitor or mast cell inhibitor that is an inhibitor of kinase activity selected from the tyrosine kinases of: c-Kit, PDGFR, LYN, FYN, or any combination thereof, especially masitinib or a pharmaceutically acceptable salt thereof, for example, 100, 200, 300, 400, or 500 mg.

In the Figures:

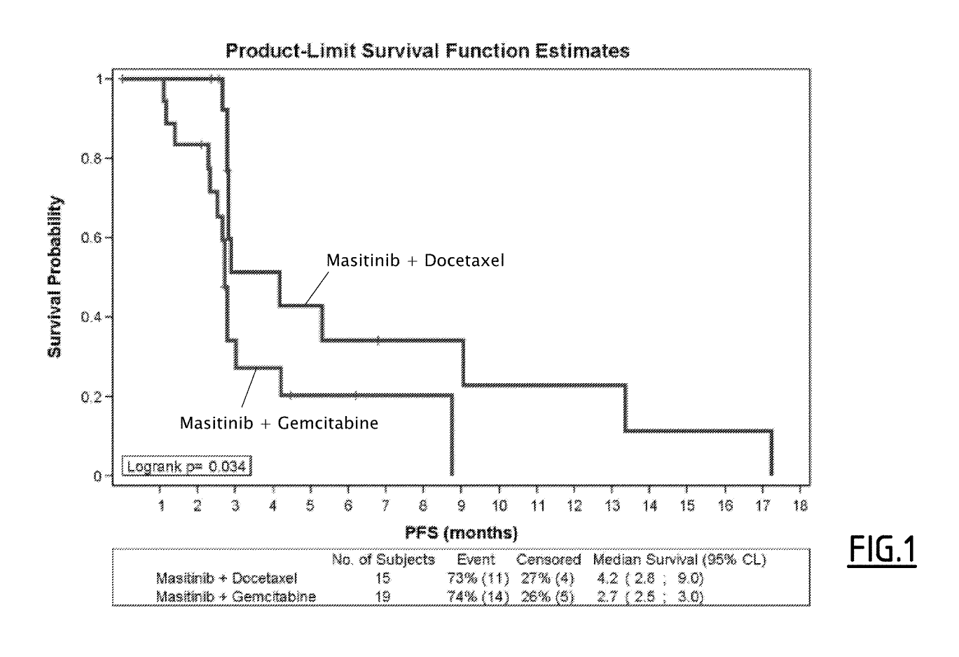

FIG. 1: Progression free survival graph according to example 1;

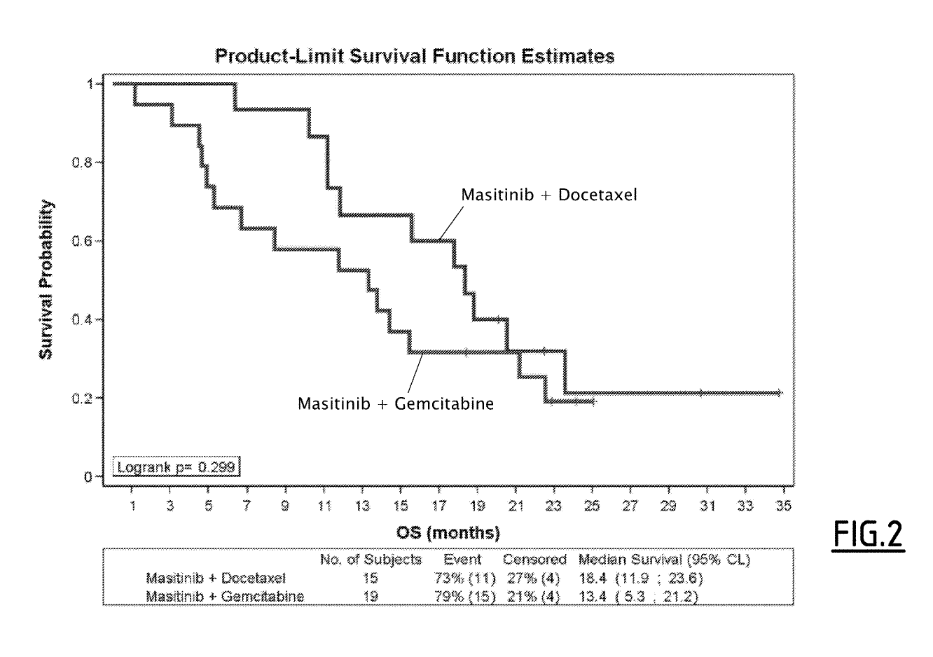

FIG. 2: Overall survival graph according to example 1.

The present invention is further illustrated by means of the following examples.

Some data presented in these examples, and also in parts of the patent Description, are in part taken from preliminary analysis and as such represent a close approximation to the final, validated dataset.

### EXAMPLES

### Example 1

### Phase ½ Study to Evaluate the Efficacy and the Safety of Masitinib in Combination with Docetaxel or with Gemcitabine in Metastatic Hormone Refractory Prostate Cancer (HRPC) in Progression after First-Line of Treatment (Second-Line Treatment)

Methodology/Study design: This was a prospective, multicenter, randomized, non comparative, open-label, 2-parallel group, phase ½ study.
Diagnosis: Patients with metastatic prostate cancer resistant to castrate levels of testosterone in progression after first-line treatment with docetaxel treatment.
Study treatment:
   Docetaxel: Patients received docetaxel at 75 mg/m² by intravenous infusion during 1 hour, once every 3 weeks (1 cycle=21 days). In addition, prednisone at 5 mg orally twice daily was administered continuously.
   Gemcitabine: Patients received at first cycle gemcitabine at 1,000 mg/m², administered by a 30 minute intravenous infusion on Day 1 and Day 8 every 21 days (1 cycle=21 days). At cycle 2, a dose escalation of gemcitabine at 1250 mg/m² was allowed if well tolerated by the patient.
   Masitinib: Patients received orally masitinib (100 and 200 mg tablets), according to tables 3 and 4 below. Optional masitinib dose reduction by 1.5 mg/kg/day increments were allowed in case of specific adverse events potentially related to masitinib for safety reasons.
Duration of treatment: Until disease progression without clinical benefit, limiting toxicity or patient consent withdrawal.
Randomization and Study Treatment Administration: A maximum of 30 patients were randomized in two groups:
   Group 1: 15 patients recieved masitinib at 9 mg/kg/day or 6 mg/kg/day (depending on the occurrence of DLT) with either full or 75% of standard dose of docetaxel and prednisone;
   Group 2: 15 patients recieved masitinib at 9 mg/kg/day or 6 mg/kg/day (depending on the occurrence of DLT) in combination with either full or 75% of initial dose of gemcitabine
Main inclusion criteria:
   Patient with histologically or cytologically confirmed metastatic prostate cancer.
   Patient with disease progression after having received: (1) hormonal therapy or surgical castration; and (2) one prior therapeutic regimen of docetaxel chemotherapy. Disease progression at trial enrolment was based on progression in at least one variable described in Table 3.

TABLE 3
Disease progression criteria at study enrolment

| Variable | Definition of Progression |
|---|---|
| PSA | Obtain sequence of rising values at a minimum of |
| | 1-week intervals |
| | Using these sequential PSA values: |
| | Progression = rising PSA level defined by 2 |
| | sequential increase |
| | above a previous lowest reference value |
| | 2.0 ng/mL minimum starting value is required at |
| | study enrolment |
| Measureable | Measureable lesions not required for study entry |
| disease | Only lymph nodes ≧ 2 cm in diameter should be |
| | used to assess for a change in size |
| | Use standard RECIST 1.1 to record soft-tissue |
| | (nodal and visceral) lesions as target or non-target |
| | Progression = use standard RECIST 1.1 criteria to |
| | evaluate soft-tissue lesions |
| Prostate/prostate | Progression = use standard RECIST 1.1 criteria; |
| bed | however, a prostate mass or recurrence in prostate |
| (primary site) | bed is not considered metastatic/progressive |
| | disease. |
| Bone | Progression = appearance of 2 or more new lesions |
| | Confirm ambiguous results by other imaging |
| | modalities (e.g., CT or MRI) |
| Other sites of | Patients with treated epidural lesions and no other |
| disease | epidural progression are eligible |

Adapted from Sher, H.I., et al. J Clin Oncol 26:1148-1159, 2008
Abbreviations:
PSA, prostate-specific antigen;
RECIST, Response Evaluation Criteria in Solid Tumors;
CT, computed tomography;
MRI, magnetic resonance imaging

Study treatment administration:
   Group 1 patients received docetaxel alone at Day 1 of Cycle 1 and only patients participating to the pharmacokinetic analysis receive masitinib at 9 mg/kg/day on Day 8 of Cycle 1. Starting from the second cycle, all group 1 patients received the combination of masitinib 9 mg/kg/day plus docetaxel as described.
   Group 2 patients, except for those participating in the PK analysis, received the combination of masitinib 9 mg/kg/day plus gemcitabine since Day 1 of Cycle 1. For Group 2 patients participating in the PK analysis, they received gemcitabine alone at Day 1 of Cycle 1. Starting from the Day 8 Cycle 1, these patients received the combination masitinib plus gemcitabine.

Optional masitinib dose reduction by 1.5 mg/kg/day increments in case of specific adverse events potentially related to masitinib were allowed for safety reasons.

TABLE 4

| | | 9 mg/kg/day | | |
|---|---|---|---|---|
| | | Daily dose | | |
| Patient's weight (kg) | | (mg) | Morning* | Evening** |
| | ≦49.9 | 400 | 200 | 200 |
| >49.9 | 61.1 | 500 | 200 | 200 + 100 |
| >61.1 | 72.2 | 600 | 200 + 100 | 200 + 100 |
| >72.7 | 83.3 | 700 | 200 + 100 | 200 + 200 |
| >83.3 | 94.4 | 800 | 200 + 100 | 200 + 200 |
| >94.4 | | 900 | 200 + 200 | 200 + 200 + 100 |

*Morning: The tables should be taken during breakfast. In case of nausea after the morning treatment intake, the administration can take place during lunch.
**Evening: The tablets should be taken during dinner.

TABLE 5

| | | 6 mg/kg/day | | |
|---|---|---|---|---|
| Patient's weight in kg | | Daily dose (mg) | Morning* (mg) | Evening** (mg) |
| | ≧41.6 | 200 | 100 | 100 |
| >41.6 | 58.3 | 300 | 100 | 200 |
| >58.3 | 74.9 | 400 | 200 | 200 |
| >74.9 | 91.6 | 500 | 200 | 200 + 100 |
| >91.6 | | 600 | 200 + 100 | 200 + 100 |

*Morning: the tablets should be taken during breakfast. In case of nausea, the administration can take place during lunch.
**Evening: the tablets should be taken during dinner.

Patient Disposition

Arm masitinib 9 mg/kg/day+docetaxel standard dose: 15 patients enrolled,

Arm masitinib+gemcitabine: 19 patients enrolled,
3 patients in masitinib 9 mg/kg/day+gemcitabine standard dose, all patients presented. Dose Limiting Toxicities and IDMC recommended reduction of dose of masitinib to 6 mg/kg/day,
16 patients in masitinib 6 mg/kg/day+gemcitabine standard dose.
Assessment of efficacy according to Progression Free Survival (PFS)

Median PFS of masitinib in combination with docetaxel and masitinib in combination with gemcitabine, were respectively 4.2 months and 2.7 months.

Median PFS of masitinib in combination with docetaxel (4.2 months) compared favorably with the median PFS of treatment registered or in development for second-line of mCRPC. The median PFS of this benchmark was 4.4 months.

Median PFS of masitinib in combination with gemcitabine (2.7 months) was lower than the median of the benchmark (4.4 months).

Progression free survival of the two treatment groups is detailed in FIG. 2.
Assessment of efficacy according to Overall Survival (OS)

Median OS was not reached for masitinib in combination with docetaxel (more than 18 months) and was 13.4 months for masitinib in combination with gemcitabine.

Median OS of masitinib in combination with docetaxel (median not reached but more than 18 months) compared favorably with the median OS of treatment registered or in development in second-line of mCRPC. The median OS of this benchmark was 14.4 months.

Median OS of masitinib in combination with gemcitabine (13.4 months) was slightly lower than the median OS of treatment registered or in development for second-line mCRPC (14.4 months).

Median OS of the two treatment groups is detailed in FIG. 3.

Median OS with masitinib in combination with docetaxel or in combination with gemcitabine could improve the existing standard survival in patients with mCRPC.
Choice of the dose

Data review of different studies of masitinib associated with chemotherapies showed that the most secure therapeutic scheme (i.e. optimized benefit-risk ratio) for the patient was masitinib 6 mg/kg/day.
Conclusions

There is a strong rationale to pursue into phase 3 the development of masitinib in mCRPC given:
The unmet medical need with an OS of 14.4 months.
That in the current phase ½ study survival with masitinib in combination with docetaxel could improve the existing standard survival in mCRPC patients after failure to single-agent docetaxel.
Masitinib's effect on inhibiting mast cells and subsequent remodeling of the tumor microenvironment.
Masitinib's effect on modulating the immune response.

Also, as docetaxel is the registered first-line chemotherapy treatment of mCPRC, docetaxel will be associated chemotherapy to masitinib, AB Science proposed to launch a phase 3 study in chemo-naïive patients, i.e., first-line chemotherapy treatment (see example 2). In order to optimize the PFS and minimize toxicity, the choice of dose of masitinib is 6 mg/kg/day.

### Example 2

### A Randomized controlled Trial to Compare the Efficacy and Safety of Masitinib in Combination with Docetaxel to Placebo in Combination with Docetaxel in First-Line Metastatic Castrate Resistant Prostate Cancer

Methodology/Study design: A prospective, multicenter, randomized, double blind, placebo-controlled, 2-parallel group, phase 3 study to compare the efficacy and safety of masitinib in combination with docetaxel to docetaxel in combination with placebo in first-line metastatic Castrate Resistant Prostate Cancer
Diagnosis: Patients with metastatic Castrate Resistant Prostate Cancer.
Study treatment: Masitinib.
Associated product and dosages:
   Docetaxel at 75 mg/m²
   Prednisone at 5 mg bid.
Duration of treatment: Until disease progression (or treatment switch to next line of treatment), death, limiting toxicity or patient consent withdrawal.
Randomization: 550 patients will be randomized in two groups:
   Group 1 (275 patients): masitinib (6 mg/kg/day)+docetaxel (75 mg/m² 6 cycles. 8 or 10 cycles possible)+prednisone according to usual practice. Group 2 (275 patients): placebo+docetaxel (75 mg/m² 6 cycles. 8 or 10 cycles possible)+prednisone according to usual practice.
Docetaxel will be combined with prednisone as usual practice. Patients will be centrally randomized to one of the two treatment group by IWRS according to minimization method.
Main inclusion criteria:
   Patient aged 18 years old, with histologically or cytologically confirmed metastatic Castrate Resistant Prostate Cancer.
   Patient with evidence of progressive metastatic disease.
Study Treatment administration: Docetaxel: 75 mg/m² by i.v. infusion during 1 hour, once every 3 weeks (1 cycle=21 days) for 6 cycles. According to patient's tolerance 8 to 10 cycles can be performed. In case of tolerance issue before 6 cycles, docetaxel can be prescribed less than 6 cycles. In addition, prednisone at 5 mg is administrated continuously orally twice daily. Masitinib/placebo: Patients will orally receive masitinib (6 mg/kg/day) or placebo. According to the initial 6 mg/kg/day dose of masitinib, the steps for the dose reduction are as follows:

| | 1st dose | 2nd dose | 3rd dose | 4^{th} dose |
|---|---|---|---|---|
| Starting dose | reduction | reduction | reduction | reduction |
| 6 mg/kg/day | 4.5 mg/kg/day | 3 mg/kg/day | 1.5 mg/kg/day | STOP |

1-23. (canceled)

24. A method for the treatment of prostate cancer, wherein said method comprises administering to a patient in need thereof at least one mast cell inhibitor.

25. The method of claim 24, wherein said mast cell inhibitor is masitinib or a pharmaceutically acceptable salt thereof.

26. The method of claim 24, wherein said patient is a human male patient.

27. The method of claim 24, wherein said patient has prostate cancer selected from the group consisting of:
advanced prostate cancer, unresectable prostate cancer, metastatic prostate cancer, castrate-resistant prostate cancer, hormone-refractory prostate cancer, androgen-independent prostate cancer, recurrent prostate cancer, and locally advanced prostate cancer.

28. The method of claim 24, wherein said patient has prostate cancer selected from the group consisting of Stage III and Stage IV prostate cancer.

29. The method of claim 24, wherein said method is for the treatment of patients with castrate-resistant prostate cancer, with or without metastatic disease, naive to chemotherapeutic agents, or after failure of at least one chemotherapeutic agent, or after failure of two or more chemotherapeutic agents.

30. The method of claim 24, wherein said method is for the treatment of patients with castrate-resistant prostate cancer, with or without metastatic disease, after failure of docetaxel-based chemotherapy or gemcitabine-based chemotherapy.

31. The method of claim 24, wherein said method is for the treatment of patients with castrate-resistant prostate cancer, with or without metastatic disease, naive to androgen-axis inhibitors, or after failure of at least one androgen-axis inhibitor, or after failure of two or more androgen-axis inhibitors.

32. The method of claim 24, wherein said method is for the treatment of patients with castrate-resistant prostate cancer, with or without metastatic disease, naive to immunotherapeutic or monoclonal antibody agents, or after failure of at least one immunotherapeutic or monoclonal antibody agent, or after failure of two or more immunotherapeutic or monoclonal antibody agents.

33. The method of claim 24, wherein said method is for the treatment of patients with advanced or recurrent prostate cancer, with or without metastatic disease, naive to androgen-deprivation agents, or after failure of at least one androgen-deprivation agent, or after failure of two or more androgen-deprivation agents.

34. The method of claim 24, wherein said method is for the treatment of patients with locally advanced prostate cancer, and said mast cell inhibitor is administered as an adjuvant to radiotherapy or in combination with at least one androgen-deprivation agent.

35. The method of claim 24, wherein said mast cell inhibitor is administered at a daily dose of 1.0 to 12.0 mg/kg/day (mg per kg bodyweight per day).

36. The method of claim 24, wherein said mast cell inhibitor is administered once or twice a day.

37. The method of claim 24, wherein said mast cell inhibitor is administered orally.

38. The method of claim 24, wherein said mast cell inhibitor is administered in combination with at least one pharmaceutically active ingredient.

39. The method of claim 24, wherein said mast cell inhibitor is administered in combination with at least one pharmaceutically active ingredient in a combined preparation for simultaneous, separate, or sequential use.

40. The method of claim 24, wherein said mast cell inhibitor is administered in combination with at least one pharmaceutically active ingredient selected from the group consisting of: (i) chemotherapeutic agents; (ii) corticosteroid agents consisting of (iia) hydrocortisone type corticosteroids and (iib) betamethasone type corticosteroids; (iii) androgen-deprivation agents; (iv) immunotherapeutic or monoclonal antibody agents; (v) or any combination thereof.

41. The method of claim 24, wherein said mast cell inhibitor is administered in combination with at least one pharmaceutically active ingredient selected from the group consisting of: (i) chemotherapeutic agents selected from docetaxel, cabazitaxel, mitoxantrone, estramustine, doxorubicin, etoposide, vinblastine, paclitaxel, carboplatin, satraplatin, vinorelbine, gemcitabine; (ii) corticosteroid agents consisting of (iia) hydrocortisone, hydrocortisone acetate, cortisone acetate, tixocortol pivalate, prednisolone, methylprednisolone, prednisone, and (iib) betamethasone, betamethasone sodium phosphate, dexamethasone, dexamethasone sodium phosphate, fluocortolone; (iii) androgen-deprivation agents selected from goserelin, leuprorelin, triptorelin, degarelix, bicalutamide, flutamide, cyproterone acetate, enzalutamide, abiraterone; (iv) immunotherapeutic or monoclonal antibody agents selected from sipuleucel-T, ipilimumab, bevacizumab; (v) or any combination thereof.

42. A pharmaceutical composition comprising a mast cell inhibitor and at least one pharmaceutically active ingredient selected from the group consisting of: (i) chemotherapeutic agents; (ii) corticosteroid agents consisting of (iia) hydrocortisone type corticosteroids and (iib) betamethasone type corticosteroids; (iii) androgen-deprivation agents; (iv) immunotherapeutic or monoclonal antibody agents; (v) or any combination thereof.

43. A kit comprising a mast cell inhibitor and at least one pharmaceutically active ingredient selected from the group consisting of: (i) chemotherapeutic agents; (ii) corticosteroid agents consisting of (iia) hydrocortisone type corticosteroids and (iib) betamethasone type corticosteroids; (iii) androgen-deprivation agents; (iv) immunotherapeutic or monoclonal antibody agents; (v) or any combination thereof.
